(19) United States
(12) Patent Application Publication
(10) Pub. No.: US 2007/0028683 A1
(43) Pub. Date: Feb. 8, 2007

(54) Apparatus and method for sensing pressure utilizing a deformable cavity

(75) Inventor: Cristian Ionescu-Zanetti, Berkeley, CA (US)

Correspondence Address: REED SMITH, LLP, TWO EMBARCADERO CENTER, SUITE 2000, SAN FRANCISCO, CA, 94111, US

(73) Assignee: The Regents of the University of California

(21) Appl. No.: 11/448,509

(22) Filed: Jun. 7, 2006

Related U.S. Application Data
(60) Provisional application No. 60/688,217, filed on Jun. 7, 2005.

(51) Int. Cl. G01F 23/00; A61B 5/02; A61B 5/00

(52) U.S. Cl. 73/299; 600/486; 600/561

(57) ABSTRACT

A pressure sensing device and method for sensing pressure utilizes a deformable cavity containing a conductive medium. Pressure changes induce deformations of the cavity, resulting in changes of conductivity, as measured by electrodes. The device may either sense pressure directly or may be used to sense the pressure in a separate cavity that is in close proximity. Since the measurements do not require electrodes in the sensing region, the device is simple to fabricate. The device also has high sensitivity, making it suitable for microfluidic or biomedical applications where a low profile and disposable device is required.

### BACKGROUND OF THE INVENTION

This application claims the benefit of U.S. Provisional Application No. 60/688,217, filed Jun. 7, 2005, entitled APPARATUS AND METHOD FOR SENSING PRESSURE UTILIZING A DEFORMABLE CAVITY, the disclosure of which is herein incorporated by reference.

1. Field of the Invention

The present invention relates generally to pressure sensing, and more particularly to an apparatus and method for sensing pressure based on the conductivity change of a conductive medium in a deformable cavity.

2. Description of the Related Art

One application of micro-electro-mechanical (MEMS) devices is local on-chip pressure sensing. For example, in micro-total analysis systems (μTAS), flow rates can be measured by monitoring local pressures on-chip. Micro-scale pressure sensors are also important for biomedical applications such as intravenous regional anesthesia, compression therapy and prosthetics. Miniaturized pressure sensors are also useful in a variety of aerospace, automotive and industrial applications.

Typically, miniaturized pressure sensors have been formed by micromachining a thin membrane on top of a cavity. The amount of deformation of the membrane in response to an applied pressure is determined by either measuring a change in capacitance across the cavity or by measuring the resistance of piezoresistive elements patterned on the membrane. These sensors can be fabricated using standard silicon processing.

On-chip flow sensing that is based on detection of pressure drops along a fluidic channel is based on similar technology. Recently the polymer elastomer polydimethylsiloxane (PDMS) has been used in the manufacture of microfluidic devices, where molding takes the place of photolithography as an inexpensive fabrication alternative. One such solution is to use a deformable PDMS diffraction grating as a sensing device, as taught by Hosokawa et al., A polydimethylsiloxane (PDMS) deformable diffraction grating for monitoring o local pressure in microfluidic devices, Journal of Micromechanics and Microengineering, 2002, 12(1), pgs. 1-6. The pressure induced deformation of the elastomer translates into a modification of the optical response of the grating, which can be used to detect pressure. However, drawbacks to this type of sensor include the large sensor area, nonlinear sensor response, and the requirements for optical readout components.

U.S. Pat. No. 4,561,450 discloses a technique to measure pressure along a tube utilizing electrodes. However, the tube is open on one end and requires three electrodes, making the technique unsuitable for many applications.

### SUMMARY OF THE INVENTION

In general, the present invention is a pressure sensing device and method for sensing pressure utilizing a deformable cavity containing a conductive medium. Pressure changes induce deformations of the cavity, resulting in changes of conductivity, as measured by electrodes. The device may either sense pressure directly or may be used to sense the pressure in a separate cavity that is in close proximity. Since the measurements do not require electrodes in the sensing region, the device is simple to fabricate. The device also has high sensitivity, making it suitable for microfluidic or biomedical applications where a low profile and disposable device is required.

In one embodiment, the present invention comprises a deformable cavity containing a conductive medium, and at least two electrodes located along a length of the cavity. As the cavity is deformed, a resistance detected by the electrodes changes, and the change in resistance is proportional to the change in pressure. The deformable cavity may be formed using a polymer elastomer, such as polydimethylsiloxane (PDMS). In another embodiment, the device may be formed using a more rigid material, where a deformable membrane or other material encloses the cavity.

A method of the present invention includes applying a pressure to a deformable cavity, the cavity containing a conductive medium, and detecting a change in resistance of the conductive medium in the deformable cavity, wherein the change in resistance is proportional to the change in pressure applied to the deformable cavity.

A further embodiment of the present invention includes forming multiple deformable cavities along the length of an apparatus, such as a catheter, in order to measure pressures along the length of the apparatus. Electrode leads may be integrally molded, such that the leads are connected to a separate pressure monitor.

### BRIEF DESCRIPTION OF THE DRAWINGS

The present invention will be readily understood by the following detailed description in conjunction with the accompanying drawings, wherein like reference numerals designate like structural elements, and in which:

FIG. 1(a) is a schematic representation of one embodiment of the present invention;

FIG. 1(b) is a cross-sectional view of the embodiment of FIG. 1(a);

FIG. 1(c) is a SEM image of an actual channel before bonding;

FIGS. 2(a)-(d) illustrate the mold fabrication steps for manufacturing a pressure sensor according to one embodiment of the present invention;

FIG. 3 is a graph of the performance of a pressure sensor formed according to the design of FIG. 1;

FIGS. 4(a) and 4(b) show the results of a simplified theoretical treatment of the single channel configuration of FIG. 1;

FIG. 5(a) is schematic representation of a second embodiment of the present invention;

FIG. 5(b) is a cross-sectional view of the embodiment of FIG. 5(a);

FIG. 5(c) is a SEM image of an actual channel formed according to the embodiment of FIG. 5(a);

FIG. 6 is a graph of the performance of a pressure sensor formed according to the design of FIG. 5(a);

FIG. 7 is an illutration of the present invention incorporated into a catheter;

FIG. 8 is an illustration of an embodiment of the present invention for sensing atmospheric or underwater pressure;

FIG. 9 is an illustration of an embodiment of the present invention incorporated into a blood pressure monitor device;

FIG. 10 is an illustration of an embodiment of the present invention configured as an accelerameter; and

FIG. 11 is an illustration of an embodiment of the present invention incorporated into a vehicle tire to monitor tire inflation.

### DETAILED DESCRIPTION OF THE INVENTION

The following description is provided to enable any person skilled in the art to make and use the invention and sets forth the best modes contemplated by the inventor for carrying out the invention. Various modifications, however, will remain readily apparent to those skilled in the art. Any and all such modifications, equivalents and alternatives are intended to fall within the spirit and scope of the present invention.

In general, the present invention is a pressure sensor that is based on a new transduction mechanism, i.e. the variation of conductivity through a fluid that is contained in a deformable cavity. One embodiment of this concept is fabricated by elastomer micromolding. The structure is very inexpensive to fabricate and can easily be integrated with existing elastomer microfluidic devices. Elastomer (i.e. PDMS) channels deform when pressure is applied to either the inside of the channel or to the outside of the channel walls. If the channel is filled with a conductive liquid medium, the electrical conductivity along the channel will be proportional to the cross sectional area of the channel and therefore directly related to channel deformation. A simple resistance measurement yields a measurement of the applied pressure. Besides ease of fabrication, a major advantage of these devices is the reduced active sensor area, which is molded out of a single material, using a single mask (electrodes can be placed far away). Thus, the present invention is suitable for use in microfluidic or biomedical applications where a low-cost and disposable sensor is required. Further, this sensing mechanism exhibits high sensitivity as compared to piezoresistive materials, without the requirements for silicon processing (except for the initial mold) or the strong temperature dependence inherent to piezoresistive devices.

When pressure is applied to a PDMS micro channel, the elastomer deforms, changing the cross-sectional area of the channel. When filled with a conductive fluid (i.e. a buffer solution), the electrical resistance of the channel changes according to the cross-sectional area, and provides a modality of monitoring the pressure inside the channel. The device may be configured to measure either a change in the internal pressure of the channel (i.e. the pressure in one or more reservoirs changes thereby changing the pressure in the channel), or a change in the external pressure (the walls or a thin membrane of the channel is deformed by pressure external to the channel itself). For other applications, the pressure sensor may be formed using silicon microchannels with a deformable membrane to sense the pressure, or other similar structures.

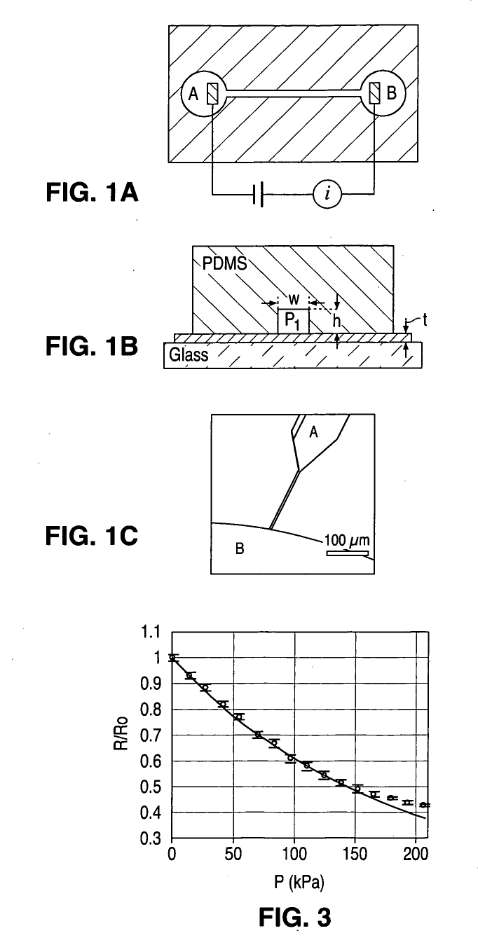

The simplest configuration for this sensing mechanism is shown in FIG. 1, with the sensor performance being presented in FIG. 3. As shown in FIG. 1(a), two Ag/AgCl electrodes A and B are formed on respective ends of a fluid-filled channel. Any conductive fluid can be used to fill the device. For purposes of the illustrated data, a conductive buffer solution was used, specifically a 2 molar KCl in phosphate buffered saline solution (pH 7.4). As illustrated in FIG. 1(b), a pressure (P₁) is applied to one end of the fluidic channel, while measuring the resistance across the channel. The other reservoir is kept at ambient pressure. FIG. 1(c) shows a SEM image of the channel before bonding to a substrate.

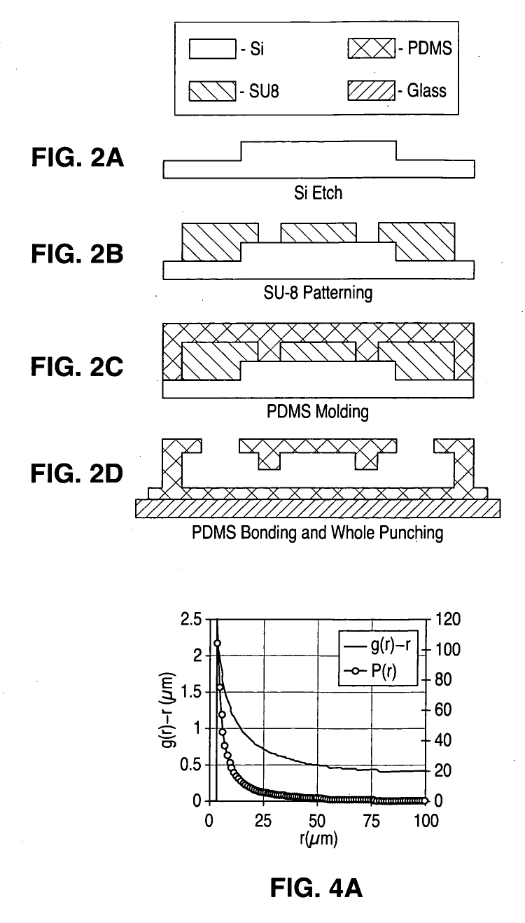

The fabrication of this sensor is depicted in FIG. 2. A silicon mold was prepared using surface micromachining techniques. First, 4 μm wide, 3.1 μm high patterns were etched in silicone by deep reactive etching, defining the mold for the narrow sensor channels (FIG. 2(a)). Then, 50 μm high patterns were added for the wide connection regions using SU-8 negative photoresist (FIG. 2(b)). For the adjacent channel geometry of FIG. 5, all channels were made with a single mold containing 10 μm high patterns. After a base and a curing agent of PDMS were mixed (1:10), the liquid mixture was then poured onto the mold and cured at 80° C. for 1 hour. SEM images of the single channel before bonding are shown in FIG. 1(c). For fluidic connections to outside tubing, 0.5 mm diameter holes were mechanically punched into the cured and detached PDMS device. The device was subsequently bonded to a thin PDMS layer which was spin casted and then cured onto a glass substrate using an oxygen plasma treatment (FIG. 2(d)). Finally, plastic tubes were connected to the reservoirs, via punched holes, to load the electrolyte solution and to apply pressure.

In FIG. 3 data is presented for the channel bonded to a thin PDMS membrane (t=10 μm) spun down onto a glass substrate. The results are presented as the variation of the unitless quantity R/R₀ as a function of pressure P; which has the advantage that the results are independent of the conductivity of the buffer solution used. For the data presented in FIG. 3, the dimensions of the channel are 4×3.1×200 μm, with a zero pressure resistance of 3.75 MΩ. The response of the sensor is almost linear in the 1-100 kPa range. Compared with piezoresistive sensor elements that change by 1-2% over the full sensor range, the channel resistance of the present device changes by 30-40% over the linear sensor range. This increased sensitivity is due to the elastic properties of PDMS, as compared to the stiff Si nitride membrane of traditional sensors.

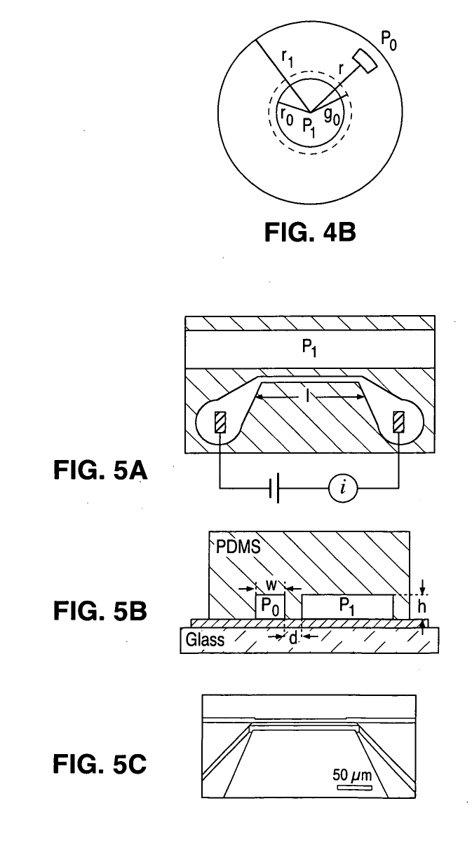

Consider the following theoretical treatment of the configuration of FIG. 1, as illustrated in FIG. 4(b). A satisfactory fit to the observed data may be obtained by considering a simple channel geometry and solving the differential equations that describe the channel deformations due to internal pressure application (for a fit, see FIG. 3). Because the deflections of the rectangular PDMS channel of FIG. 1 are large (Δr≈r₀), the final conformation will be approximately cylindrical, so the idealized geometry of FIG. 4b is used. In this idealized geometry, g(r) denotes the new position of a shell element originally located at position r, so the deformation is equal to g(r)−r. When pressure P₁ is applied to the interior of the channel, each section of material of crossectional area dA=dr·rdθ moves from its initial radial position r to a new position g(r). The force balance equations for the azimuthal and radial directions are as follows:

$$\begin{matrix}- \frac{1}{E} \cdot \frac{ⅆ P}{ⅆ g} = \frac{g ( r ) - r}{{r}^{2}} & ( 1 ) \\ \frac{ⅆ g}{ⅆ r} = 1 - \frac{P ( r )}{E} & ( 2 )\end{matrix}$$

where E is the Young's modulus of PDMS. Eliminating P provides:
gg′=rg′+r²g′ (3)
Taking into account the boundary conditions g′(r₀)=1−P₁/E and g′(r₁)=1 set by the pressure inside and outside the cylinder (FIG. 4b), the equation can be solved numerically, taking the inner radius to be r₀=3.5 μm, the outer radius as r₁=10 μm, and the inside pressure P₁=210 kPa. The outer edge of our device is much farther, but increasing the outer radius further doesn't affect the results significantly. The choice of inner radius r₀ does not significantly influence the resulting unitless quantity R/R₀, as long as r₀<<r₁. Since pressure is only applied to one end of the channel (reservoir A), one can assume a linear pressure distribution along the channel length, dropping to P₀=0 at reservoir B.

Fitting E, the Young's modulus, to the data produces a value of E=310 kPa, which agrees well with values reported in the literature. The deformation and the pressure distribution inside the PDMS cylinder are plotted in FIG. 4(a). Note that the pressure inside the elastomer decays to within 1% of the pressure applied channel interior in the first 20 μm from the inner radius, making this an absolute pressure transducer since the deformation will be independent of the pressure outside the device as long as r₁>>r₂.

Traditionally, sensor sensitivity is evaluated by considering the measured voltage difference across a Wheatstone bridge per voltage excitation of the bridge. If the resistors are all of the same value (R₁=R₂=R₃=Rₓ), the voltage output change with respect to changes in the variable voltage Rₓ works out to be dVₒᵤₜ/dRₓ=Vᵢₙ/4Rₓ, so dVₒᵤₜ/dP=dRₓ/dP×Vᵢₙ/4Rₓ. Assuming that the bridge is balanced at P=0, which gives Rₓ=3.75 MΩ. In the linear range, the sensor sensitivity is 16.7 kΩ/kPa, or an equivalent sensitivity of 148 μV/V/mmHg. This is about an order of magnitude higher than the sensitivities reported for silicon micromachined piezoresistive pressure sensors, which range from 10-20 μV/V/mmHg [see, Melvas et al., A temperature compensated dual beam pressure sensor, Sensors and Actuators a-Physical, 2002. 100(1): p. 46-53; Bistue, G., et al., A micromachined pressure sensor for biomedical applications, Journal of Micromechanics and Microengineering, 1997. 7(3): p. 244-246]. For the present design, decreasing the channel dimensions provides a way of increasing sensor sensitivity dR/Rₜₒₜₐₗ at the expense of sensor range. The resistance can be measured using either a Wheatstone bridge or an ohm meter.

Since pressure is applied at one end of the channel only, the pressure is also a measure of outward flow through the channel for known channel dimensions. The relationship between the pressure drop and the flow rate has been worked out [see K. Foster and G. A. Parker, Fluidics: Components and Circuits, Wiley Interscience, New York, 1970) and for the channel dimensions of FIG. 1 it yields:

$$Q = \frac{6 μ L}{{wh}^{3}} \qquad ( 4 )$$

Where μ is the viscosity of water, L the channel length (L=200 μm) and w and h the channel dimensions. This setup can easily measure pressure changes of about 5 kPa, resulting in a flow rate detection limit of 0.50 nL/s. Shrinking the channel dimensions would reduce the cross-sectional area and increase the fluidic resistance, resulting in greater sensitivity to flow rates. For example, for a 2×2 μm channel the detection limit should decrease to 0.13 nL/s. By comparison, the highest sensitivity flow sensors based on heat measurement can detect flows as low as 0.028 nL/s.

For lab-on-a-chip applications it is advantageous to be able to measure pressure in a channel of interest that is filled with an arbitrary liquid or gas, while segregated from the sensing channel. For such applications, local pressure inside the fluidic channel of interest can be measured by placing a sensing channel in close proximity to, but separated from, the main channel by a thin membrane. Pressure changes in the main channel will result in a deformation of the membrane and changes in the resistance (cross-sectional area) of the test channel. This setup is schematically represented in FIG. 5. In this configuration, the sensing channel is kept at ambient pressure (P₀), while a pressure or a flow rate is applied to the adjacent fluidic channel (P₁, f₁), as shown in FIG. 5(b). A light microscope image of a finished device is shown in FIG. 5(c).

In addition to the sensing channel dimensions w and h, an important parameter for this configuration is the thickness of the membrane separating the two channels, d. Measurements are performed by applying pressure P₁ to the main channel while the sensing channel is kept at the reference pressure P₀, equal to atmospheric pressure in this case. The flow rate in the main channel can be obtained by placing sensing elements at different points along the channel and measuring the pressure differential along the length of the channel.

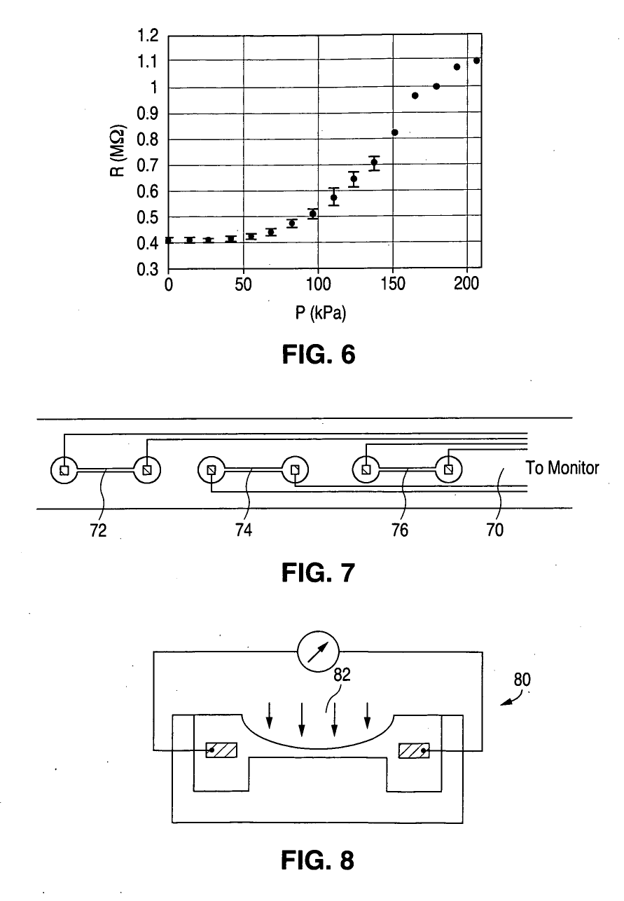

Performance data for the two channel configuration is shown in FIG. 6. Error bars represent the standard deviation of the data, and no error bars indicate that only one data run was performed in the high pressure range. For the device tested, sensor dimensions were h=5 μm, w=10 μm, d=5 μm, and l=200 μm. For this configuration, the sensitivity is lower in the range of 0-56 kPa, and increases in the linear range of 56-175 kPa. In this range, using an initial resistance Rₓ=0.41 MΩ, we obtain a sensitivity of 0.038 MΩ/psi, or 442 μV/V/mmHg in the Wheatstone bridge configuration. These results can be improved upon by optimizing the sensor geometry for particular applications.

In summary, the present invention is a new micro-scale pressure sensing mechanism. The principal advantages of this sensing mechanism are very simple and inexpensive fabrication for disposable packages, and simple readout electronics, which basically consist of any resistance measurement setup. The sensitivity is about an order of magnitude higher than that of existing piezoresistive pressure sensor technology. Also, since the sensor is based on ionic conductivity, the temperature dependence is relatively low, chiefly determined by the temperature stability of the elastomer used. This eliminates the need for temperature compensation circuitry. In addition, the sensor can be easily integrated with existing elastomer microfluidic devices.

Two potential problems include electrode degradation through the depletion of one type of ion (i.e. Cl⁻) from one of the electrodes, and the fluid leakeage into the PDMS bulk after a long exposure under pressure. The first problem can be solved by periodical reversal of the bias applied to the sensing channel, therefore replenishing the ionic content of the electrodes. The second problem can be addressed by covering the PDMS structure with a gas/buffer impermeable coating.

There are many possible specific applications for the present sensor. One application of the invention is for local pressure and flow rate monitoring in lab-on-a-chip systems, where accurate dispensing of small fluid volumes is necessary. In general, it could also be applied to the more traditional MEMS markets such as medicine (disposable blood pressure monitors) and automotive pressure monitors and accelerometers (reduced to practice by attaching a weight to a pressure monitoring device). For example, as illustrated in FIG. 7, the present invention may be incorporated into a catheter 70 to measure pressures at various points along the catheter. Separate sensors 72, 74, 76 may be formed along the length of the catheter 70 to measure pressures at various points, with the electrode leads connected to thin wires which connect to an external pressure monitor (not shown).

The present invention may also be configured to function as a barometer 80 to measure external pressure. As shown in FIG. 8, a thin membrane 82 is exposed to ambient pressure, and any changes in pressure are perceived as a change in the resistance reading.

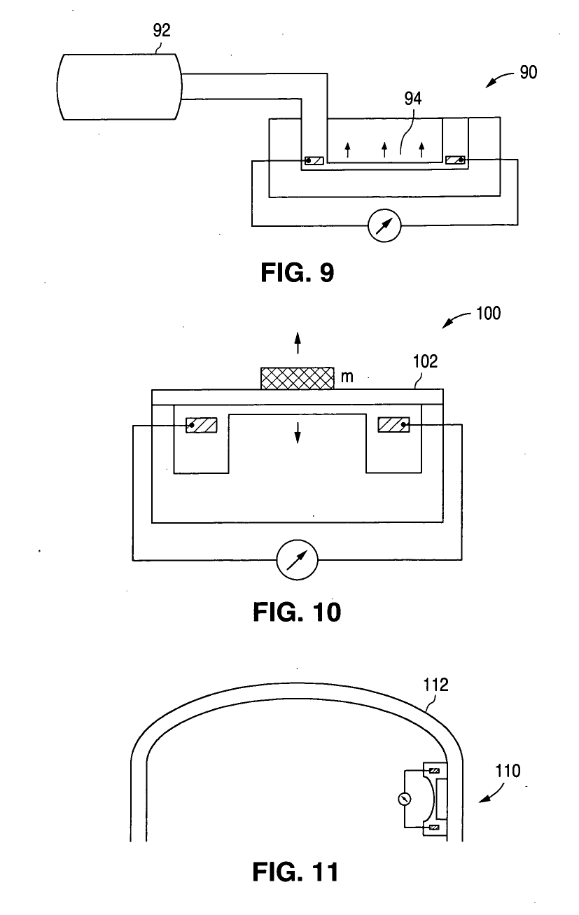

As shown in FIG. 9, the present pressure sensor may be incorporated into a blood pressure device 90. As the pressure builds in the cuff 92, the pressure can be sensed directly in the channel 94.

For automotive applications, the pressure sensor may be fashioned as an accelerometer 100 as shown in FIG. 10. A weight m is applied to a thin membrane 102. Sudden changes in acceleration are detected as sudden changes in the resistance reading. Such an accelerometer 100 may be applied for air bag deployment or other similar applications.

Similarly, the pressure sensor 110 could be incorporated into an automobile tire 112 as shown in FIG. 11, in order to measure tire pressure. The pressure measurement could be sent to a notification system via a wireless sensor, to alert a driver if the tire is under or over inflated.

Those skilled in the art will appreciate that various adaptations and modifications of the just-described preferred embodiments can be configured without departing from the scope and spirit of the invention. Therefore, it is to be understood that, within the scope of the appended claims, the invention may be practiced other than as specifically described herein.

What is claimed is:

1. A pressure sensing device comprising:
an enclosed deformable cavity containing a conductive medium; and
at least two electrodes located along a length of the cavity,
wherein as the cavity is deformed, a resistance detected by the electrodes changes.

2. The pressure sensing device of claim 1, wherein the device is formed of a polymer elastomer.

3. The pressure sensing device of claim 2, wherein the polymer elastomer is polydimethylsiloxane.

4. The pressure sensing device of claim 1, wherein the cavity is formed having a thin membrane to sense an external pressure.

5. The pressure sensing device of claim 4, wherein the cavity is formed in proximity to a separate cavity having a pressure to be measured.

6. The pressure sensing device of claim 1, wherein a change in internal pressure cause the cavity to deform, and the detected change in resistance is a measure of the change in the internal pressure.

7. The pressure sensing device of claim 1, wherein a change in external pressure causes the cavity to deform, and the detected change in resistance is a measure of the change in the external pressure.

8. The pressure sensing device of claim 1, wherein the device detects air pressure or underwater pressure.

9. The pressure sensing device of claim 1, wherein the device detects blood pressure.

10. The pressure sensing device of claim 1, wherein the device detects acceleration and/or deceleration of a vehicle.

11. The pressure sensing device of claim 1, wherein the device detects a level of inflation of a vehicle tire.

12. The pressure sensing device of claim 1, wherein the resistance is measured by an ohm meter.

13. The pressure sensing device of claim 1, wherein the resistance is one element of a Wheatstone bridge.

14. A method of detecting a change in pressure, the method comprising:
applying a pressure to an enclosed deformable cavity, the cavity containing a conductive medium; and
detecting a change in resistance of the conductive medium in the enclosed deformable cavity, wherein the change in resistance is proportional to the change in pressure applied to the enclosed deformable cavity.

15. The method of claim 14, wherein the cavity is formed of a polymer elastomer.

16. The method of claim 14, wherein the deformable cavity is formed in proximity to a separate cavity having a pressure to be measured.

17. The method of claim 14, wherein electrodes are formed at opposite ends of the cavity to detect the change in resistance.

18. A system for measuring a plurality of pressures along the length of an apparatus, the system comprising:
at least two enclosed deformable cavities formed at positions along the length of the apparatus, the deformable cavities containing a conductive medium; and
at least two electrodes located along a length of each cavity,
wherein as each cavity is deformed, a resistance detected by the respective electrodes changes.

19. The system of claim 18, wherein the apparatus is a catheter.

20. The system of claim 18, wherein the cavities are formed in proximity to a separate cavity having a pressure to be measured.

21. The system of claim 20, wherein the apparatus is a catheter.

22. A method for measuring a rate of fluid flow in a channel, the method comprising:
placing an enclosed deformable cavity, the cavity containing a conductive medium, in proximity to the channel, such that increased or decreased fluid flow in the channel changes deformation of the enclosed deformable cavity; and
detecting a change in resistance of the conductive medium in the enclosed deformable cavity, wherein the change in resistance is proportional to the change in the rate of fluid flow in the channel.
